(19) United States
(12) Patent Application Publication
(10) Pub. No.: US 2009/0252684 A1
(43) Pub. Date: Oct. 8, 2009

(54) Determination of tissue-specific profile of amino acid requirements form the relationship between the amounts of tRNAs for individual amino acids and the protein-bound amino acid profile of the tissue

(76) Inventors: Robert R. Wolfe, Little Rock, AR (US); John Troup, Plymouth, MN (US); Nicolaas E. P. Deutz, Little Rock, AR (US)

Correspondence Address: LEWIS, RICE & FINGERSH, LC;ATTN: BOX IP DEPT., 500 NORTH BROADWAY, SUITE 2000, ST LOUIS, MO, 63102, US

(21) Appl. No.: 12/332,311

(22) Filed: Dec. 10, 2008

Related U.S. Application Data
(60) Provisional application No. 61/012,743, filed on Dec. 10, 2007.

(51) Int. Cl. A61K 49/00; A61K 31/198; A61P 35/00

(52) U.S. Cl. 424/9.2; 514/563

(57) ABSTRACT

Systems and methods by which optimal profiles of amino acids for specific tissues can be determined. The results of these systems and methods can then be sued to generate medical nutrition products targeting specific tissues either to enhance growth of specific tissues by providing complementary amino acids in a nutritional supplement or pharmaceutical composition form. They can also be used to generate similar medical nutrition products to inhibit growth of particular tissues, such as those that are cancerous

### CROSS REFERENCE TO RELATED APPLICATION(S)

This application claims benefit of and priority to U.S. Provisional Patent Application Ser. No. 61/012,743 filed Dec. 10, 2007 the entire disclosure of which is herein incorporated by reference.

### BACKGROUND

1. Field of the Invention

The invention relates to systems and methods for the determination of optimal profiles of amino acids to optimally support the synthesis of protein in specific tissues and such resultant profiles.

2. Description of the Related Art

Amino acids are potent regulators of muscle protein synthesis. This response has been attributed in part to an amino acid-induced stimulation of eukaryotic initiation factor-2 (eIF2) protein kinase phosphorylation-mediated signaling. A general effect of amino acid concentrations on protein synthesis through p70 (s6k) signaling transduction pathway has also been reported. Regardless of the state of the initiation process, sufficient amino acid precursors must be available for an activated initiation process to be reflected in an increased rate of synthesis of protein. Whereas the intracellular pool provides the precursor amino acids for muscle protein synthesis, transfer ribonucleic acid (tRNA) charged with amino acids serves as the ultimate precursors for protein synthesis. tRNA functions to activate amino acids and recognize codons in messenger RNA (mRNA) for protein synthesis. Each amino acid is charged with the appropriate tRNA by an activating aminoacyl-tRNA synthase, which is specific for each amino acid as well as for the corresponding tRNA. However, little information is available regarding the in vivo charging of tRNA, particularly in muscle.

Limited studies suggest that tRNAs are generally highly charged with amino acids under normal physiological conditions. However, the tRNAs for only a few specific amino acids have been investigated. Charging of leucyl-tRNA has been found to be close to complete in the livers of rats, even after one or two days of starvation. However, there are no data available regarding the extent of charging of the tRNAs for most amino acids in liver. Only indirect information regarding the extent of charging of tRNA in muscle is available. It has been reported that the Kms for the synthase enzyme responsible for the charging of leucyl-tRNA in rat muscle is well below the normal intracellular concentration. To our knowledge, there is no information regarding the relation between the Kms and corresponding concentrations of intracellular amino acids other than leucine. Further, there are no data quantifying the actual charging of tRNA for any amino acid in muscle or estimates of individual tissue requirements for the relative proportion of amino acids

### SUMMARY

Because of these and other problems in the art, described herein are systems and methods for determining optimal patterns, or profiles, of amino acids to optimally support the synthesis of protein in specific tissues has not previously been described. The invention includes a process by which optimal profiles for specific tissues can be determined, thereby enabling production of medical nutrition products targeting specific tissues. The process involves determining the amount of each individual amino acid bound to tRNA in a tissue and comparing the resulting profile with the profile of the protein-bound amino acid pool. Tissue requirements are determined to be the profile of ingested amino acids necessary to have the pattern of the tRNA-bound coincide with the pattern of the tissue protein-bound pool of amino acids.

Transfer ribonucleic acid (tRNA) charged amino acids are direct precursors of protein synthesis. Therefore, the amount and profile of amino acids in the aminoacyl-tRNA pool may be closely related to the rate of protein synthesis in the tissue. This study was designed to compare the aminoacyl-tRNA pools in liver and muscle, two distinct tissues with different rates of protein synthesis. Liver and muscle samples were taken from 6 rabbits and aminoacyl-tRNA was isolated with sequential acid-phenol: chloroform extraction, followed by total RNA and tRNA purification. Amino acids in the aminoacyl-tRNA pool were measured by HPLC after deacylation. Liver contained 4-fold more tRNA than muscle (585±120 vs 132±11 μg of tRNA per gram of tissue, p<0.001). Overall tRNA charging was also greater in liver (14.22±4.42 nmol of amino acids per mg of tRNA) than in muscle (7.00±1.76 nmol of amino acids per mg of tRNA) (p<0.05). The greater availability and charging efficiency of tRNA in liver as compared to muscle may influence the extent to which amino acid precursor availability regulates protein synthesis in these two tissues.

There is described herein, among other things, a method for determining an optimal profile of amino acids to support the synthesis of protein in a specific tissue; the method comprising: providing an intravenous infusion comprising a plurality of amino acids to an animal; determining an amount of each of said amino acids in said plurality which is bound to tRNA in a tissue; determining an amount of each of said amino acids in a protein-bound amino acid pool; and comparing said amounts of each of said amino acids in said plurality which is bound to tRNA in a tissue to said amount of each of said amino acids in a protein-bound amino acid pool to determine a profile of amino acids for said tissue.

There is also described herein, a method for inhibiting the growth of a cancerous tumor, the method comprising: determining an optimal profile of amino acids for said tissue; providing a nutritional product comprising amino acids which disrupts said profile.

There is also described herein a composition comprising: a plurality of amino acids; and a carrier vehicle; wherein the amount of each of said amino acids in said composition is selected to cause a pattern of tRNA-bound amino acids in an animal coincide with a pattern of a tissue protein bound pool of amino acids in said animal.

### BRIEF DESCRIPTION OF THE FIGURES

FIG. 1 shows electrophoresis of tRNA sample on 15% denaturing polyacrylamide gel with 8 M urea. 1 μg of sample isolated from rabbit liver (A) and muscle (B) was stained with ethidium bromide. The RNA was visualized by UV transilluminator. There are clear tRNA, 5S rRNA, and 5.8S rRNA bands.

FIG. 2 shows total aminoacyl-tRNA pools in muscle and liver. * significantly different from muscle, p<0.01.

FIG. 3 shows the relation between aminoacyl tRNAs of essential amino acids and corresponding contributions to protein. A. Liver and Albumin; B. Muscle. Values are percent contribution to essential amino acid pool (valine and methionine were not measured).

FIG. 4 shows a table of Amino Acid Concentrations

FIG. 5 shows a Table of Proportional contributions of amino acids to plasma, tRNA and tissue-bound pools of amino acids

### DETAILED DESCRIPTION

Discussed below are systems and methods for determining optimal patterns, or profiles, of amino acids to optimally support the synthesis of protein in specific tissues has not previously been described. In an embodiment there is provided a process by which optimal profiles for specific tissues can be determined, thereby enabling production of medical nutrition products targeting specific tissues. The process involves determining the amount of each individual amino acid bound to tRNA in a tissue and comparing the resulting profile with the profile of the protein-bound amino acid pool. Tissue requirements are determined to be the profile of ingested amino acids necessary to have the pattern of the tRNA-bound coincide with the pattern of the tissue protein-bound pool of amino acids.

This specific profile of ingested amino acids can be provided as part of a pharmaceutical composition or nutritional supplement. When the composition is in the form of a food (or nutritional) supplement, the latter comprises for example a palatable base which acts as a vehicle for administering the composition to an individual and which can mask any unpleasant taste or texture of the composition. The food supplement may contain any one or several nutrients including drugs, vitamins, herbs, hormones, enzymes and/or other nutrients. The nutritional supplement may contain plural parts, where each of the plural parts is chronologically appropriate for its scheduled time of consumption.

When the composition is in the form of a pharmaceutical composition, it can be administered in conventional form for oral administration, e.g. as tablets, lozenges, dragees and capsules utilizing a carrier vehicle in the same manner as a supplement. However, in certain cases it may be preferred to formulate the composition as an oral liquid preparation such as a syrup. The medicament can also be administered parenterally, e.g. by intramuscular or subcutaneous injection, using formulations in which the medicament is employed in a saline or other pharmaceutically acceptable, injectable composition.

The reverse logic could be used to formulate a nutritional product pr pharmaceutical composition that is designed to preferentially limit growth of specific tissues. For example, by disrupting the optimal pattern of the amino acid bound to tRNA, one could potentially limit the growth of cancerous tumors.

Tablets and capsules for oral administration are usually presented in a unit dose, and contain conventional excipients such as binding agents, fillers, diluents, tabletting agents, lubricants, disintegrants, colourants, flavourings, and wetting agents. The tablets may be coated according to well-known methods in the art.

Suitable fillers for use include, mannitol and other similar agents. Suitable disintegrants include starch derivatives such as sodium starch glycollate. Suitable lubricants include, for example, magnesium stearate.

These solid oral compositions may be prepared by conventional methods of blending, filling, tabletting or the like. Repeated blending operations may be used to distribute the active agent throughout those compositions employing large quantities of fillers. Such operations are, of course, conventional in the art.

Oral liquid preparations may be in the form of, for example, aqueous or oily suspensions, solutions, emulsions, syrups, or elixirs, or may be presented as a dry product for reconstitution with water or other suitable vehicle before use. Such liquid preparations may contain conventional additives such as suspending agents, for example sorbitol, syrup, methyl cellulose, gelatin, hydroxyethylcellulose, carboxymethyl cellulose, aluminium stearate gel or hydrogenated edible fats, emulsifying agents, for example lecithin, sorbitan monooleate, or acacia; non-aqueous vehicles (which may include edible oils), for example, almond oil, fractionated coconut oil, oily esters such as esters of glycerine, propylene glycol, or ethyl alcohol; preservatives, for example methyl or propyl p-hydroxybenzoate or sorbic acid, and if desired conventional flavouring or colouring agents.

Oral formulations further include controlled release formulations, which may also be useful in the practice of this invention. The controlled release formulation may be designed to give an initial high dose of the active material and then a steady dose over an extended period of time, or a slow build up to the desired dose rate, or variations of these procedures. Controlled release formulations also include conventional sustained release formulations, for example tablets or granules having an enteric coating.

Nasal spray compositions are also a useful way of administering the pharmaceutical preparations of this invention to patients such as children for whom compliance is difficult. Such formulations are generally aqueous and are packaged in a nasal spray applicator, which delivers a fine spray of the composition to the nasal passages.

Suppositories are also a traditionally good way of administering drugs to children and can be used for the purposes of this invention. Typical bases for formulating suppositories include water-soluble diluents such as polyalkylene glycols and fats, e.g. cocoa oil and polyglycol ester or mixtures of such materials.

For parenteral administration, fluid unit dose forms are prepared containing the compound and a sterile vehicle. The compound, depending on the vehicle and the concentration, can be either suspended or dissolved. Parenteral solutions are normally prepared by dissolving the compound in a vehicle and filter sterilising before filling into a suitable vial or ampoule and sealing. Advantageously, adjuvants such as a local anaesthetic, preservatives and buffering agents are also dissolved in the vehicle.

Parenteral suspensions are prepared in substantially the same manner except that the compound is suspended in the vehicle instead of being dissolved and sterilised usually by exposure to ethylene oxide before suspending in the sterile vehicle.

Advantageously, a surfactant or wetting agent is included in the composition to facilitate uniform distribution of the compound of the invention.

As is common practice, the compositions will usually be accompanied by written or printed directions for use in the medical treatment concerned.

Described herein is a new approach in order to fully understand the possible role of precursor availability in controlling the rate of protein synthesis in vivo. Rather than focus on the percentage charging of tRNA, we have instead measured the total availability of charged tRNA, since this should be the more relevant parameter when considering precursor availability. Also, rather than focus on a single amino acid, we have measured the availability of 16 aminoacyl-tRNAs, since the synthesis of a protein requires adequate availability of all of the component amino acids. We have used this methodology to determine differences in the total pool sizes of charged aminoacyl-tRNAs in liver and muscle. Since the fractional synthetic rate of liver protein is several-fold greater than that of muscle, if there is a relation between precursor availability and protein synthesis, it should be reflected by a significantly greater aminoacyl-tRNA pool in liver than in muscle.

### Methods

Tissue Sample Preparation

Male New Zealand White rabbits (Myrtle's Rabbitry; Thompson Station, Tenn.), weighing between 4-5 kg, were used for this study. The rabbits were housed in individual cages and acclimated with Lab Rabbit Chow HF 5326 (Purina Mills, St. Louis, Mo.) and water for a week. After an overnight fast, an amino acid mixture (10% Travasol; Baxter Healthcare, Deerfield, Ill.) was infused intravenously at 0.5 ml-1·kg·h−1 under general anesthesia. One hundred milliliters of the Travasol solution contains 730 mg leucine, 600 mg isoleucine, 580 mg lysine hydrochloride, 580 mg valine, 560 mg phenylalanine, 480 mg histidine, 420 mg threonine, 400 mg methionine, 180 mg tryptophan, 2.07 g alanine, 115 g arginine, 1.03 g glycine, 680 mg proline, 500 mg serine, and 40 mg tyrosine. The infusion was given to maintain constant amino acid concentrations throughout the study. Three hours after the start of the infusion fresh tissues were taken from the liver and adductor muscle of hindlimbs. Tissue samples were rinsed quickly in ice-cold saline (0.9% sodium chloride solution), frozen immediately in liquid nitrogen, and were stored in −80° C. for further processing.

The anesthetic and surgical procedures have been described in previous publications (15,16). In brief, the rabbits were anesthetized with ketamine and xylazine. Polyethylene catheters (PE-90; Becton-Dickinson, Parsippany, N.J.) were inserted in the right femoral artery and vein through an incision in the groin. The arterial line was used for blood collection and monitoring of heart rate and mean arterial blood pressure; the venous line was used for infusion of amino acids. A tracheal tube was placed via tracheotomy. The free end of the tracheal tube was placed in an open hood that was connected to an oxygen supply line so that the rabbits spontaneously breathed room air enriched with oxygen.

This protocol complied with NIH guidelines and was approved by the Animal Care and Use Committee of The University of Texas Medical Branch at Galveston.

### Aminoacyl-tRNA Isolation

Aminoacyl-tRNA was isolated from 5 g of tissue using the A mirVana™ miRNA Isolation kit (Ambion, Inc. Austin, Tex.) (17). Ten 0.5 g pieces of tissue were processed separately and the tRNA pooled. In order to eliminate free amino acids that were not bound with tRNA, the tRNA pellet was washed by Wash Solution (provided by the mirVana™) three times and resolved in nuclease-free water. The amount and purity of tRNA were assessed by measuring the absorbance at UV-260 nm (A260) and the ratio of A260/A280 in a DU650 spectrophotometer (Beckman Coulter, USA). After determining the A260 value by multiplying the spectrophotometer reading by the dilution factor, the RNA concentration was calculated as described in the mirVana™ mi RNA Isolation Instruction Manual. As a check for purity, the isolated tRNA fraction was analyzed by electrophoresis on denaturing 15% polyacrylamide gel with 8 M urea.

### Aminoacyl-tRNA Deacylation

Aminoacyl-tRNA was deacylated in 0.12M KOH (pH 9.0) at 37° C. for 1 hour, as reported by Davis et al (18). Amino acids were separated from tRNA by acidification with 0.5M HCL to pH 2.0 and centrifuged for 30 minutes at 3,000 g. The supernatant, which contained amino acids, was dried under nitrogen gas stream. The dried amino acids were reconstituted in 1.0 ml of 1 M acetic acid and 20 μl of a standard solution containing norvaline (10 nmol/ml) and β-aminobutyric acid (30 nmol/ml). The amino acids were purified through a cation exchange column (Ag 50W-X8 resin, 200-400 mesh, H+ form, BioRad Laboratories, Richmond, Calif.) and dried in a speed vacuum concentrator (AES 2010-220, Savant Instruments, Inc. Holbrook, N.Y.) at room temperature. In order to measure the amino acids isolated from the aminoacyl-tRNA sample, 20 μl of acetonitrile and 80 μl of H2O was added to the dried amino acids. After setting on ice for 30 min, 60 μl of the sample was passed through an ultrafree-MC centrifugal filter (5,000 NMWL filter units, Millipore, Bedford, Mass.) by centrifuging at 4,000 g and 4° C. for 4 hours. The filtrates were stored at −80° C. before HPLC analysis.

### Isolation of Blood Free Amino Acids and Protein-Bound Amino Acids

To determine plasma free amino acid concentrations, 50 μl of arterial plasma was precipitated with 100 μl of cold acetonitrile. An aliquot of 100 μl of standard solution containing norvaline (10 nmol/ml) and β-aminobutyric acid (30 nmol/ml) was added. After centrifugation, the supernatant was transferred to centrifugal filtration vials (5000 NMWL filter unit; Millipore, Bedford, Mass.) and centrifuged at 3,000×g for 5 hours. The clear solution that had passed through the filter was used for HPLC analysis of amino acid concentrations. Tissues (liver or muscle) were hydrolyzed by 3 mL of 6 M HCl at 110° C. for 24 hours. After hydrolysis, the tissue-bound amino acids of tissues were processed in the same manner as amino acids deacylated from aminoacyl-tRNA.

### HPLC Analysis of Amino Acid Concentrations

The amino acid concentrations from plasma, the deacylated tRNA and hydrolyzed muscle and liver proteins were measured by reverse phase high performance liquid chromatography (HPLC) equipped with fluorescence detector using o-phthaldehyde (OPA) derivative (18). The concentration of each individual amino acid was obtained from the chromatogram peak area comparison with standard. The amount of each individual amino acid bound with tRNA was calculated by: measured concentration (nmol/ml)×0.12 ml/the recovery factor, where 0.12 ml is the volume of each sample.

### Method Validation

tRNA recovery test: To evaluate tRNA recovery, the following experiment was repeated four times: 0.4 g of fresh rabbit liver was homogenized in 4 ml of Lysis/Binding buffer. The homogenate was evenly divided into two aliquots. 150 μg of tRNA (Ribonucleic acid, Type XI: from bovine liver, 50 Units, 17.2 A260 units/mg, Sigma-Aldrich, Inc. Cat. no. R-4752) in 100 μl H2O was added to aliquot B while 100 μl of nuclease-free H2O was added into aliquot A. They were then mixed and incubated 10 min on ice; and the same aminoacyl-tRNA isolation procedure as described above was followed for both aliquots. The tRNA recovery was evaluated by the difference between the amounts of tRNA in each aliquot, divided by the amount of tRNA added.

Determination of possible contamination of tRNA pellet with free amino acids. The following two tests were performed to determine if the measured amino acids were all derived from the aminoacyl-tRNA pool rather than the free amino acids in the tissue fluid.

In the first test, 0.9 μl of L-[4,5-³H]-leucine (5.66 TBq/nmol, 153 Ci/mmol, Cat no. TRK510, Amersham Biosciences) was mixed with the homogenization buffer before it was added to the frozen muscle tissue. The tissue was processed by the same RNA isolation procedure as described above. At each step of isolation and washing, 100 μl of solution was taken and measured by a LKB 1219 liquid scintillation counter (Wallac Oy, Turku, Finland).

In the second test, 265 nmol of exogenous phenylalanine was added to 5 ml of the homogenization buffer before adding the frozen muscle tissue. All the discarded solutions were collected for measurement of phenylalanine in the solutions by HPLC. The concentration of free phenylalanine in the final discarded washing solution was compared to that of the corresponding value from the deacylation of charged tRNA.

Reproducibility and reliability of aminoacyl-tRNA isolation. To determine the reproducibility of the method, ten pieces of muscle samples (5 g each) from five rabbits were processed for tRNA isolation and purification on two different days. The amount and purity of tRNA was assessed by the absorbance at UV-260 nm (A₂₆₀) and the ratio of A_{260/280}.

### Statistical Analysis

All values were expressed as mean±SEM. Statistical analysis between paired samples from muscle and liver were performed with two-tailed t-test. Significance was accepted at the level of p<0.05.

### Results

### Method Validation

Recovery of tRNA. Recovery was calculated as the difference between the samples before (A) and after (B) the standard was added; the average values for aliquot A and B were 100.06±0.46 and 182.14±1.35 μg. Since the amount of standard added was 150.43±0.74 μg, the average recovery was (B−A)/150.43=54.6±0.60%. Calculation of tRNA concentrations were therefore corrected for recovery.

Contamination of tRNA-bound amino acids with free amino acids. The total dpm (disintegrations per minute, radioactivity unit) of ³H-leucine added to the extraction was 23199.5±236.7 dpm. After washing 3 times with the wash solution from the kit, the dpm in the discarded washing solution and tRNA pellet both declined to the background level. Therefore, there was no detectable free amino acids in the tRNA pellet. However, the absence of detectable radioactivity does not exclude the possible presence of a small undetectable amount of free amino acids. Therefore, a second test was performed to compare the amounts of amino acids in the aminoacyl-tRNA pool and in the discarded washing solution. Results showed that the concentration of phenylalanine in the third washing solution was 0.08 nmol/ml, which was an insignificant percent of the starting concentration (53 mmol/ml of free phenylalanine in the homogenization buffer. The contamination of the isolated tRNA with free amino acids was therefore considered to be acceptable.

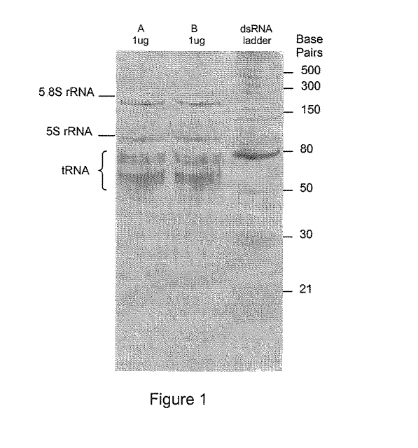

Reproducibility and reliability of purification of tRNA. Repetitive analysis of muscle samples from 5 rabbits showed that there was consistent tRNA yield, with a coefficient of variation of 7.5%. The ratio of A₂₆₀/A₂₈₀ was 1.99±0.01, indicating a high purity of the tRNA isolated as well. Visualization of the results of electrophoresis indicated only minor contamination with rRNA (FIG. 1).

Concentration of tRNA in tissues. Liver contained 585±120 mg of tRNA per gram of tissue, as compared to 132±11 mg/gm in muscle (p<0.001).

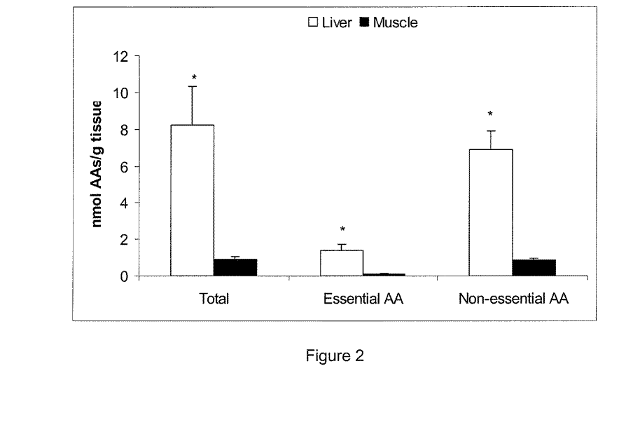

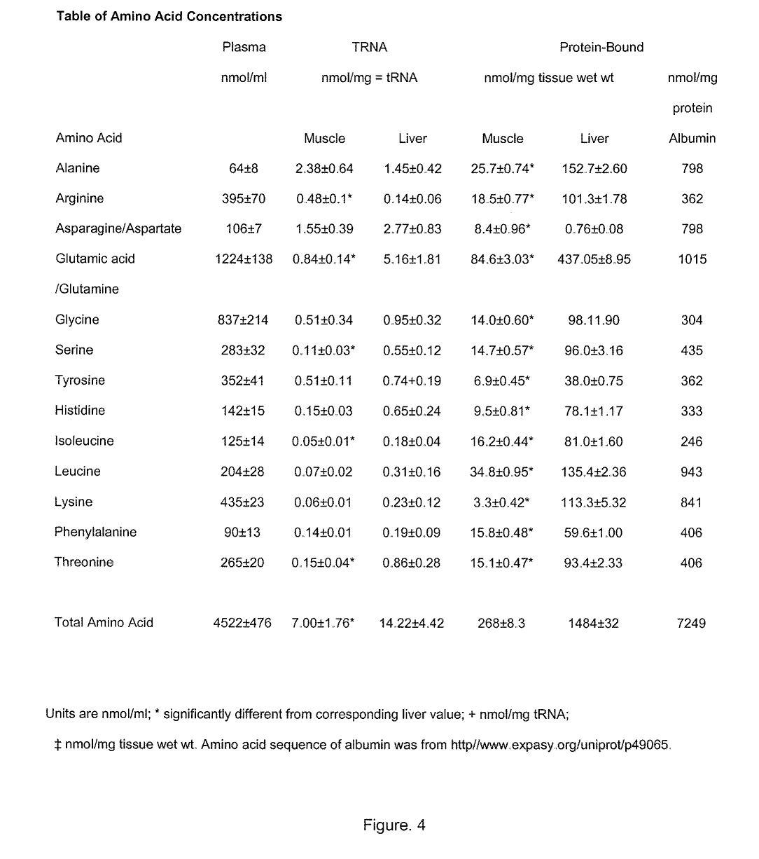

Aminoacyl tRNA pools. The relative charging of tRNA was greater in liver (14.21±4.42 mmol amino acids/mg of tRNA) than in muscle (7.00+1.5 mmol amino acids/mg of tRNA) (p<0.05) (FIG. 4). Coupled with the differences in tRNA in the two tissues, this meant that the total amount of amino acids in the aminoacyl-tRNA pools was several-fold greater in liver than muscle. Total aminoacyl-tRNA charged with essential amino acids was 1.43±0.38 nmol amino acids/g liver, which was greater (p<0.01) than the corresponding value in muscle (0.08±0.03 nmol/g). The non-essential amino acids in the tRNA pool were also greater in liver (6.87±2.83 mmol/g of liver vs 0.84±0.14 nmol/g of muscle) (p<0.05) (FIG. 2).

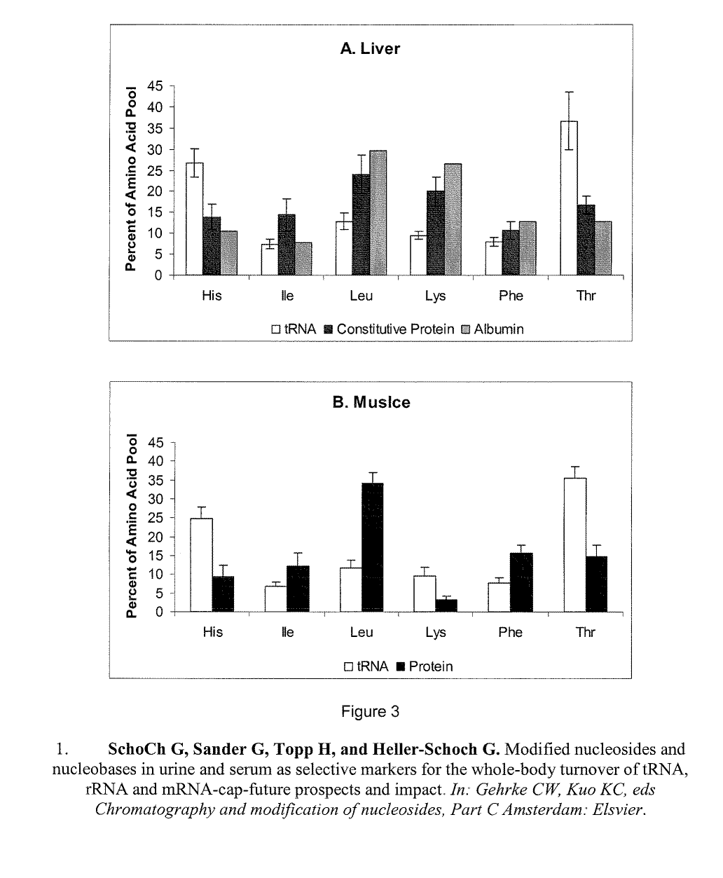

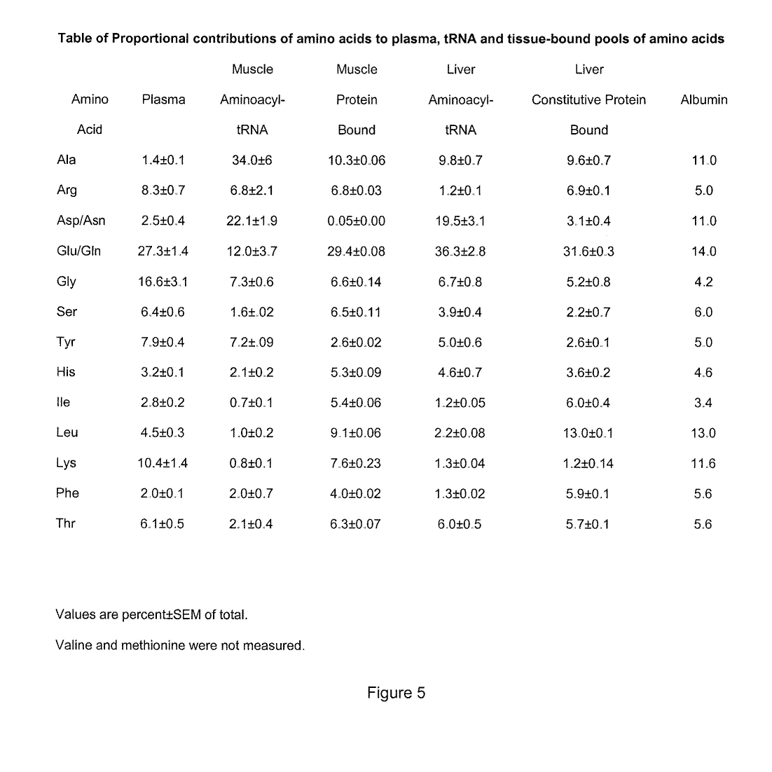

Amino acid concentrations. Measured concentrations of amino acids in plasma, and the aminoacyl tRNA pool, and protein-bound pools in liver and muscle are shown in FIG. 4. The distribution of amino acids in albumin, the major protein synthesized in the liver and secreted, is also shown. The values are expressed as percent contribution to the corresponding pool listed in FIG. 5. The profiles of the various pools differed. The ratio of essential amino acids (EAAs), other than valine and methionine (which were not measured), to the non-essential amino acids (NEAAs) was highest in the protein-bound pools and lowest in the tRNA pools. The profile of amino acids in plasma bore little resemblance to the pattern in the tRNA pools, and similarly the profiles in the aminoacyl tRNA pools did not closely parallel those of the protein-bound pools. Certain discrepancies were particularly marked. For example, aspartate/asparagine comprised approximately 20% of the aminoacyl tRNA pools in both tissues, but a minimal amount of either amino acid was present in the protein bound pool of muscle or liver, although albumin contains 11% of aspartate/asparagine. The glutamine/glutamate aminoacyl tRNA pool in muscle was relatively smaller than the protein-bound pool, but the values corresponded more closely in liver. Among the essential amino acids, the proportions of isoleucyl- and leucy-tRNAl were lower than their corresponding contribution to muscle protein whereas the proportions of histidyl- and lysyl-tRNA were greater than their contribution to muscle protein. (FIG. 3). In the liver, the greatest discrepancies between the charged tRNA and protein bound amino acid were seen with leucine and threonine. In the case of leucine, the proportional contribution of the aminoacyl tRNA pool of EAAs was only 12.8±2.3%, as compared to 24.1±4.2% in the protein-bound EAA pool. Threonine, on the other hand, comprised 35.6±3.2% of the aminoacyl-tRNA pool of EAAs, but only 16.6% of the protein-bound EAA pool. As in muscle, histidine was proportionately more abundant in the aminolacyl-tRNA pool than in the protein-bound pool, and the converse was true for lysine. The distribution of essential amino acids in the constitutive proteins of liver was similar to that of albumin (FIG. 3).

### Discussion

The importance of tRNA in linking amino acid availability with the process of protein synthesis is well recognized, yet little research has been directed to the in vivo role of tRNA availability in the regulation of the rate of protein synthesis. To our knowledge this is the first report of the amounts of tRNA in different tissues, as well as the size of the aminoacyl tRNA pools. These values are of relevance, since the aminoacyl tRNA pool is the immediate precursor of protein synthesis. Our principal finding was that there was approximately 4 times the tRNA in the liver, per gram of tissue, than in muscle, and that under the conditions of this experiment the aminoacyl-tRNA pool was approximately 8 times greater in liver. The charging percent of aminoacyl-tRNA was also determined in total tRNA. We further found that whereas the proportionate availability of certain species of aminoacyl tRNA was well matched with the corresponding contribution of that amino acid to the protein in that tissue, in other cases there were wide discrepancies.

To our knowledge this paper reports tissue levels of tRNA, as well as the individual aminoacyl tRNA pools, for the first time. The three primary types of RNA molecules are mRNA, rRNA and tRNA. tRNA comprises about 12% of total cellular RNA. In most papers investigating the aminoacyl-tRNA pool, the total RNA has been isolated, rather than the tRNA specifically. In this paper we have isolated tRNA from the mRNA and most of the rRNA. This isolation method enabled for the first time the quantification of the amount of charged tRNA in tissues. Because we have used a new method, it is pertinent to examine its validity.

The most likely source of error in the measurement of the aminoacyl tRNA pool is contamination of the charged tRNA with free amino acids from the intracellular pool. There are two potential sources of contamination: exogenous contamination from the reaction system, and endogenous amino acid contamination from free amino acids. A blank control group was run to exclude exogenous interference. The radioactivity assay confirmed that there was no free amino acid in the purified aminoacyl tRNA fraction. In addition, the second contamination test involving the addition of exogenous phenylalanine showed that after the washing steps the retention of exogenous amino acid was minimal. Further, the replicate analysis of ten samples of muscle showed the procedure for measuring the amount of tRNA to be consistent and reproducible, and the gel electrophoresis results documented good isolation of tRNA from mRNA and fairly complete isolation from rRNA.

It is generally believed that the charging of tRNA is not rate limiting for protein synthesis. However, the data directly supporting that conclusion are limited. Studies have shown that charging of leucyl-tRNA in liver is close to complete, even in the fasting state, and that deprivation of a single amino acid from the diet (isoleucine) does not affect the charging of the corresponding aminoacyl-tRNA that in the brain. However, a simultaneous assessment of the charging of several aminoacyl-tRNAs in these tissues has not been undertaken. Data are even more limited in muscle. The Km for the synthetase to form leucyl-tRNA is well below the intracellular concentration, and therefore it has been considered that the charging of tRNA is always complete in muscle as well. The extent of charging of tRNA in muscle with leucine, or any other amino acid, has not been measured.

The data presented in the current paper do not directly address the issue of the percent charging of the individual tRNAs. However, if it assumed that the molecular weight of tRNA is 2.5×10⁴, and the measured liver tRNA charging was 14.21±4.42 mmol amino acids/mg of tRNA and muscle charging was 7.00±1.5 nmol amino acids/mg of tRNA, it can be calculated that charging was 35.6% in liver and 17.5% in muscle These data are inconsistent with previous reports of close to complete charging of leucyl-tRNA in liver, and muscle. The low extent of charging of total tRNA could be due to low charging of aminoacyl tRNAs other than leucyl-tRNA). For example, a portion of the discrepancy between liver and muscle may be explained by the amount of glutamate/glutamine bound to tRNA in liver as compared to muscle. In any case, the low values for total charging that we have observed give reason to examine in the future the extent of charging of individual tRNAs.

There is indirect evidence that the extent of charging of tRNA does not control the rate of muscle protein synthesis. Thus, we have shown that muscle protein synthesis was stimulated during the infusion of amino acids into normal human subjects at rates sufficient to increase plasma concentrations within the normal physiological range, even though the intracellular concentrations of free amino acids remained either unchanged or slightly depressed. Since the amino acids involved in charging of muscle tRNA apparently come from the intracellular pool, our results are consistent with the notion that a stimulation of muscle protein synthesis above the normal basal rate is not mediated by an increase in tRNA charging. On the other hand the potential role of the total availability of charged tRNA in regulating the rate of protein synthesis must be considered. Anderson first suggested that the total amount of tRNA may play an important role in regulating the rate of protein synthesis in 1969 based on results from E. coli. However, to our knowledge this concept has not been extended to the in vivo situation. In this study we found that the liver contained 4 fold more tRNA per gram of tissue than muscle, and the amount of amino acids per gm of liver tRNA was also greater than in muscle, meaning that the amount of amino acids in the aminoacyl-tRNA pool in liver was approximately 8 fold greater than in muscle. Garlick et al reported than in rat liver the average fractional synthetic rate of protein was 50% per day, which was approximately 7 fold greater than that of muscle (7.2% per day). It thus appears that amount of tRNA in a tissue, combined with the extent of charging, may be directly related to the rate of protein synthesis in that tissue. This suggests that not only is amino acid availability important, but also factors regulating the amount of tissue tRNA are important in determining the rate of synthesis of protein in a give tissue.

The profile of amino acids in the aminoacyl tRNA and protein-bound pools reveal some interesting discrepancies which may relate to regulatory roles of individual amino acids. A number of studies, including our own, have pointed to a potential regulatory role of leucine in both whole body as well as muscle protein synthesis. In both liver and muscle the amount of leucyl-tRNA was found to be disproportionately lower than the relative contribution of leucine to protein produced in that tissue (FIG. 3). Therefore, it is possible that the regulatory role of leucine stems from the relatively low amount of leucyl-tRNA in the basal state, thereby making leucine availability rate-limiting. This speculation is consistent with our work in identifying the optimal formulation of amino acids to stimulate muscle protein synthesis, as we have found that a mixture containing a disproportionate amount of the branched chain amino acids to be advantageous as compared to the distribution of amino acids in a high quality protein such as whey. Consequently, it may be that a formulation directed towards balancing the relationships between the aminoacyl tRNA pools and the distribution of amino acids in protein produced in that tissue may be a means by which to specifically target the production of certain proteins.

### CONCLUSION

We have presented a method which enables quantification of the amount of all aminoacyl-tRNAs in a tissue. We found that the total amount of aminoacyl-tRNA differ's by several fold in the liver and muscle, and this difference roughly parallels the differences between the respective tissue protein synthetic rates. Our method provides a means by which to study not only the role of the aminoacyl-tRNA pool in regulating muscle protein synthesis, but also factors regulating the size of the aminoacyl-tRNA pool itself.

1. A method for determining an optimal profile of amino acids to support the synthesis of protein in a specific tissue; the method comprising:
providing an intravenous infusion comprising a plurality of amino acids to an animal;
determining an amount of each of said amino acids in said plurality which is bound to tRNA in a tissue
determining an amount of each of said amino acids in a protein-bound amino acid pool;
comparing said amounts of each of said amino acids in said plurality which is bound to tRNA in a tissue to said amount of each of said amino acids in a protein-bound amino acid pool to determine a profile of amino acids for said tissue.

2. A method for inhibiting the growth of a cancerous tumor, the method comprising:
determining an optimal profile of amino acids for said tissue;
providing a nutritional product comprising amino acids which disrupts said profile.

3. A composition comprising:
a plurality of amino acids; and
a carrier vehicle;
Wherein the amount of each of said amino acids in said composition is selected to have a pattern of tRNA-bound amino acids in an animal coincide with a pattern of a tissue protein bound pool of amino acids in said animal.
